(19) United States
(12) Patent Application Publication
(10) Pub. No.: US 2019/0002821 A1
(43) Pub. Date: Jan. 3, 2019

(54) METHODS AND COMPOSITIONS FOR DETACHING ADHERENT CELLS

(71) Applicant: PLURISTEM LTD., Haifa (IL)

(72) Inventor: Lior Raviv, Kfar-Monash (IL)

(21) Appl. No.: 16/026,199

(22) Filed: Jul. 3, 2018

Related U.S. Application Data
(60) Provisional application No. 62/528,169, filed on Jul. 3, 2017.

(51) Int. Cl. C12N 5/00; C12M 1/26; C12N 5/073; C12N 5/077

(52) U.S. Cl. CPC C12N 5/00; C12M 33/00; C12N 2509/10; C12N 5/0604; C12N 5/0652; C12N 5/0605

(57) ABSTRACT

This disclosure is directed to methods and systems for harvesting cells.

### RELATED APPLICATIONS

This application claims the benefit of U.S. Provisional Application No. 62/528,169, filed Jul. 3, 2017. The contents of the aforementioned application are hereby incorporated by reference in its entirety.

### FIELD OF THE TECHNOLOGY

The present disclosure relates to systems and methods for harvesting cells.

### BACKGROUND

Some cells grow well in suspension, while others require surface adherence for growth and division. The latter type has classically been grown (expanded) in monolayers on glass or plastic surfaces (2D matrices), in tissue cultures dishes, multi-well plates, or similar devices.

More recently, adherent cells have been expanded on three-dimensional (3D) carriers and matrices. Such matrices can include porous, non-woven or woven fibrous carriers, as well as sponge-like materials, that can be placed in a packed bed inside a bioreactor. These carriers are often used for the production and collection of secreted proteins, while the cells remain attached to the matrix, rather than for the culture of cells that are ultimately removed and used as therapeutic agents. Examples of such carriers are Fibra-Cel® Disks (New-Brunswick).

Adherent cells grown on 2D matrices are typically removed for passaging by enzymatic treatment (e.g. trypsin), followed by gentle agitation using a pipet or the like. However, such methods are ineffective for cells attached to fibrous 3D matrices, since in the latter situation, cells are attached more tightly. The problem is further complicated by the fact that robust physical agitation can compromise the viability of cells removed from the matrix and/or their potency for downstream usages, particularly in the presence of, or shortly after contact with, proteases. WO/2012/140519 to Barak Zohar et al describes certain solutions; however, many of the solutions in the art cannot be scaled up to larger bioreactors or suffer from other problems, such as foaming, inconsistent harvest efficiency, and/or viability of cells in the resultant suspension.

A different problem occurs with harvest from grooved, rigid 3D carriers, e.g. carriers containing multiple 2D surfaces extending from the exterior towards the interior thereof, e.g. those described in WO/2014/037862 to Eytan Abraham et al. The rigid exterior shields the cells from sheer forces exerted on the carriers, necessitating a different magnitude and dynamic of forces to detach the cells while maintaining their viability. Furthermore, the cells need to be flushed from the inner spaces of these carriers, in order to be efficiently collected into a pharmaceutical suspension.

### SUMMARY OF THE DISCLOSURE

Aspects of the disclosure relate to systems and methods that allow more efficient harvesting of cells from 3D carriers.

In some embodiments, there is provided a method of detaching adherent cells from fibrous, three-dimensional (3D) carriers, comprising the steps of (a) incubating the adherent cells with an agent that disrupts adhesion of the adherent cells to the carrier; and (b) subjecting the 3D carriers to a rotary motion while the 3D carriers are submerged in an aqueous solution. In certain embodiments, the 3D carriers are disposed within a bioreactor chamber. Those skilled in the art will appreciate that fibrous carrier typically have a flexible structure.

Also provided herein is a method of detaching adherent cells from grooved, rigid, 3D carriers, comprising the steps of: (a) incubating the adherent cells with an agent that disrupts adhesion of the adherent cells to the carrier; and (b) subjecting the 3D carriers to a rotary motion while the 3D carriers are submerged in an aqueous solution. PCT Publication Number WO/2014/037862 to Eytan Abraham et al describes some embodiments of rigid carriers. These carriers can comprise multiple 2D surfaces, wherein these multiple 2D surfaces are configured to support monolayer growth of eukaryotic cells over at least a majority of the 2D surfaces.

Additional embodiments consistent with principles of the disclosure are set forth in the detailed description which follows or may be learned by practice of methods or use of systems or articles of manufacture disclosed herein. It is understood that both the foregoing general description and the following detailed description are exemplary and explanatory only, and are not restrictive of the disclosure as claimed. Additionally, it is to be understood that other embodiments may be utilized and that electrical, logical, and structural changes may be made without departing form the spirit and scope of the present disclosure.

### BRIEF DESCRIPTION OF THE DRAWINGS

The accompanying drawings, which are incorporated in and constitute a part of this specification, illustrate several embodiments of the disclosure and together with the description, serve to explain the principles of the disclosure. In the drawings:

FIG. 1A is a perspective view of a carrier (or “3D body”), according to an exemplary embodiment. FIG. 1B is a perspective view of a carrier, according to another exemplary embodiment. FIG. 1C is a cross-sectional view of a carrier, according to an exemplary embodiment.

FIG. 2 is a perspective view of a system for growing and harvesting cells, according to an exemplary embodiment.

FIG. 3A is a perspective view of a system in an open configuration, according to an exemplary embodiment. FIG. 3B is a perspective view of an impeller, according to an exemplary embodiment.

FIG. 4 is a perspective view of a system for growing and harvesting cells in an opened position, according to another exemplary embodiment.

FIG. 5A is a perspective view of a system for growing and harvesting cells in an opened position, according to another exemplary embodiment. FIG. 5B is a perspective view of a rotating cylinder of the system of FIG. 5A, according to an exemplary embodiment.

FIG. 6 is a diagram of a bioreactor that can be used to expand adherent cells.

### DESCRIPTION OF THE EMBODIMENTS

Reference will now be made in detail to the present embodiments of the disclosure, examples of which are illustrated in the accompanying drawings.

In this application, the use of the singular includes the plural unless specifically stated otherwise. Also in this application, the use of “or” means “and/or” unless stated otherwise. Furthermore, the use of the term “including,” as well as other forms, such as “includes” and “included,” are not limiting. Any range described herein will be understood to include the endpoints and all values between the end points.

Certain embodiments of the systems and devices of the present disclosure are configured for harvesting cells. Certain embodiments of these vessels are configured to operate with various shaped and sized carriers, including but not limited to spherical, cylindrical, cubical, hyperrectangular, ellipsoid, and polyhedral shapes, having a variety of sizes, as specified herein. In some embodiments, these carriers allow growth (proliferation) of eukaryotic cells. Reference herein to “growth” of cells is synonymous with expansion of a cell population.

The phrase “two-dimensional culture” refers to a culture in which the cells are exposed to conditions that are compatible with cell growth and allow the cells to grow in a monolayer, which is referred to as a “two-dimensional culture apparatus”. Such apparatuses will typically have flat growth surfaces, in some embodiments comprising an adherent material, which may be flat or curved. Non-limiting examples of apparatuses for 2D culture are cell culture dishes and plates. Included in this definition are multi-layer trays, such as Cell Factory™ manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (substrate), in some embodiments comprising an adherent material, which is present in the 3D culture apparatus, e.g. the bioreactor. Certain, non-limiting embodiments of 3D culturing conditions suitable for expansion of adherent stromal cells are described in PCT Application Publ. No. WO/2007/108003 to Ora Burger et al, which is fully incorporated herein by reference in its entirety. Typically, cells growing on a 3D substrate grow outside of the confines of a monolayer. Carriers that enable 3D culture are referred to herein as 3D carriers.

In some embodiments, there is provided a method of detaching adherent cells from fibrous, three-dimensional (3D) carriers, comprising the steps of (a) incubating the adherent cells with an agent that disrupts adhesion of the adherent cells to the carrier; and (b) subjecting the 3D carriers to a rotary motion while the 3D carriers are submerged in an aqueous solution. In certain embodiments, the 3D carriers are disposed within a bioreactor chamber. Those skilled in the art will appreciate that fibrous carriers typically have a flexible structure.

In certain embodiments, the 3D carriers comprise an adherent material. In some embodiments, the adherent material is fibrous, which may be, in more specific embodiments, a woven fibrous matrix, a non-woven fibrous matrix, or either. In still other embodiments, the material exhibits a chemical structure such as charged surface groups, which allows cell adhesion, e.g. polyesters, polypropylenes, polyalkylenes, polyfluorochloroethylenes, polyvinyl chlorides, polystyrenes, polysulfones, cellulose acetates, and poly-L-lactic acids. In more particular embodiments, the material may be selected from a polyester and a polypropylene. Those skilled in the art will appreciate that fibrous matrices are typically porous.

Alternatively or in addition, the 3D carriers comprise a fibrous material, optionally an adherent, fibrous material, which may be, in more specific embodiments, a woven fibrous matrix, a non-woven fibrous matrix, or either. Non-limiting examples of fibrous carriers are New Brunswick Scientific Fibracel® carriers, available commercially from of Eppendorf Inc, Enfield, Conn., and made of polyester and polypropylene; and BioNOC II carriers, available commercially from CESCO BioProducts (Atlanta, Ga.) and made of PET (polyethylene terephthalate). In certain embodiments, the referred-to fibrous matrix comprises a polyester, a polypropylene, a polyalkylene, a polyfluorochloroethylene, a polyvinyl chloride, a polystyrene, or a polysulfone. In more particular embodiments, the fibrous matrix is selected from a polyester and a polypropylene.

In certain embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively or in addition, the material is fibrous, which may be, in more specific embodiments, a woven fibrous matrix, a non-woven fibrous matrix, or any type of fibrous matrix. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. Other embodiments include Matrigel™, an extra-cellular matrix component (e.g., Fibronectin, Chondronectin, Laminin), and a collagen. In more particular embodiments, the material may be selected from a polyester and a polypropylene. Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids.

In certain embodiments, the 3D carriers and the aqueous solution are disposed within a chamber, and the rotary motion is imparted by protruding objects projecting radially from a central axis of the chamber. In more specific embodiments, the described chamber is cylindrical. In other embodiments, the described chamber is a variation of a cylindrical shape, e.g. an irregular cylinder, whose cross-sectional area is not constant along the axis of the cylinder. In other embodiments, the cylinder is an elliptic cylinder, parabolic cylinder, or hyperbolic cylinder, which refer, respectively, to a cylinder whose cross section is an ellipse, parabola, or hyperbola. In still other embodiments, the cylinder is an oblique cylinder, namely a cylinder the two circular end planes are parallel to each other, but unlike the right circular cylinder wherein the lateral surface (the curved surface) is not perpendicular to the end planes. The described protruding objects, in some embodiments, extend radially from an axial element that is configured to rotate and is aligned with the central axis of the cylinder or variation thereof. In certain embodiments, axial element is itself cylindrical or a variation thereof, having a small diameter than the diameter of the chamber. “Axial” in this context refers to a line connecting the centers of the bases of a cylinder or a similar shape.

The described protruding objects, are in certain embodiments, spoke-like projections. In other embodiments, the protruding objects may be rod-shaped. Alternatively or in addition, there may be 10-50, 10-40, 10-30, 15-50, 15-40, 15-30, 15-25, 17-23, 20-30, or 22-28 spokes extending radially from a central axis. In still other embodiments, the protruding objects and axial element may together exhibit a helix conformation.

In still other embodiments, the length of the protruding objects (perpendicular to the chamber axis) is 50-90%, 50-95%, 50-80%, 40-95%, 40-90%, 40-80%, 40-70%, or 50-70% of the inner cross-sectional radius of the chamber, wherein the measurement extends from the external surface of the axial element to the inner surface of the chamber wall. Thus, the protruding objects extend the indicated percentage of the distance from the external surface of the axial element to the inner surface of the chamber wall.

Alternatively or in addition, the radius of the protruding objects (parallel to the chamber axis) is 0.5-5%, 0.5-4%, 0.5-3%, 0.5-2%, 0.5-1.5%, 0.4-5%, 0.4-4%, 0.4-3%, 0.4-2%, 0.4-1.5%, 0.3-5%, 0.3-4%, 0.3-3%, 0.3-2%, or 0.3-1.5% of the chamber axis.

In some embodiments, the protruding objects are rotated in a continuous rotary motion. In more specific embodiments, the rotary motion is between 100-400 rpm; in other embodiments 100-350 rpm; in other embodiments 100-300 rpm; in other embodiments 120-300 rpm; in other embodiments 100-250 rpm; in other embodiments 120-250 rpm; in other embodiments 50-250 rpm in other embodiments 60-250 rpm in other embodiments 80-250 rpm; or in other embodiments 150-250 rpm. Alternatively or in addition, the protruding objects are rotated for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes.

In still other embodiments, the protruding objects are subjected to rotational force (relative to a central axis of the chamber) in an oscillating fashion. In some embodiments, the protruding objects are moved in an oscillating rotary motion for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes. Alternatively or in addition, the frequency of the oscillation may be once per 1-10 seconds; 1-15 seconds; 1-20 seconds; 1-30 seconds; 1-45 seconds; 1-60 seconds; 2-10 seconds; 2-15 seconds; 2-20 seconds; 2-30 seconds; 2-45 seconds; 2-60 seconds; 3-10 seconds; 3-15 seconds; 3-20 seconds; 3-30 seconds; 3-45 seconds; 3-60 seconds; 5-10 seconds; 5-15 seconds; 5-20 seconds; 5-30 seconds; 5-45 seconds; 5-60 seconds; 10-20 seconds; 10-30 seconds; 10-45 seconds; or 10-60 seconds.

The maximal angular velocity of the described rotary motion (which is, in some embodiments, continuous rotary motion, or is, in other embodiments, oscillating rotary motion) is, in certain embodiments, 3-30, 3-25, 3-20, 3-15, 3-10, 4-30, 4-25, 4-20, 4-15, 4-10, 5-30, 5-25, 5-20, 5-15, 5-10, 6-30, 6-25, 6-20, 6-15, 6-10, 8-30, 8-25, 8-20, 8-15, or 8-10 rotations per minute (rpm).

Each of the aforementioned embodiments of the number, geometry, and arrangement of the protruding objects; and the velocity, duration, type, and frequency (in the case of oscillating motion) of the rotary motion may be freely combined.

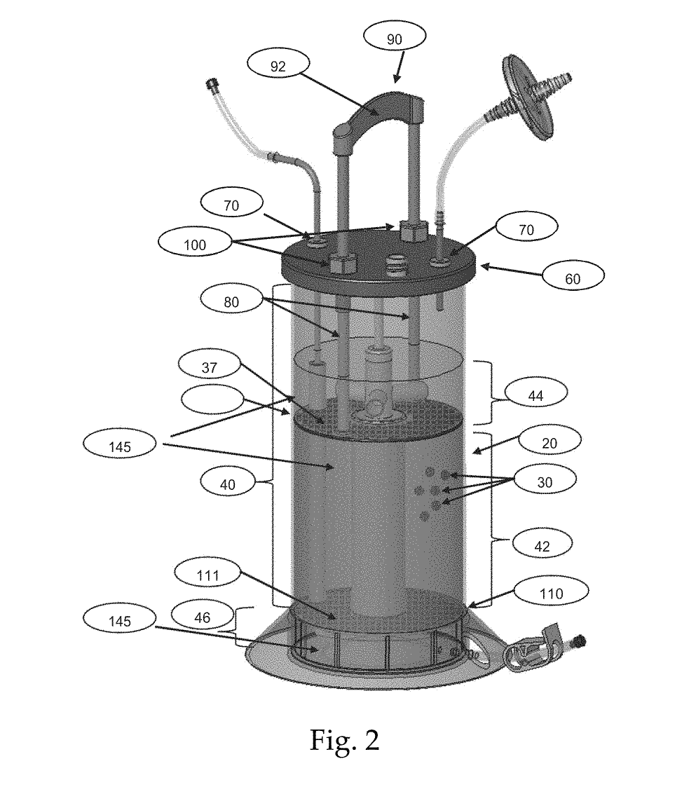

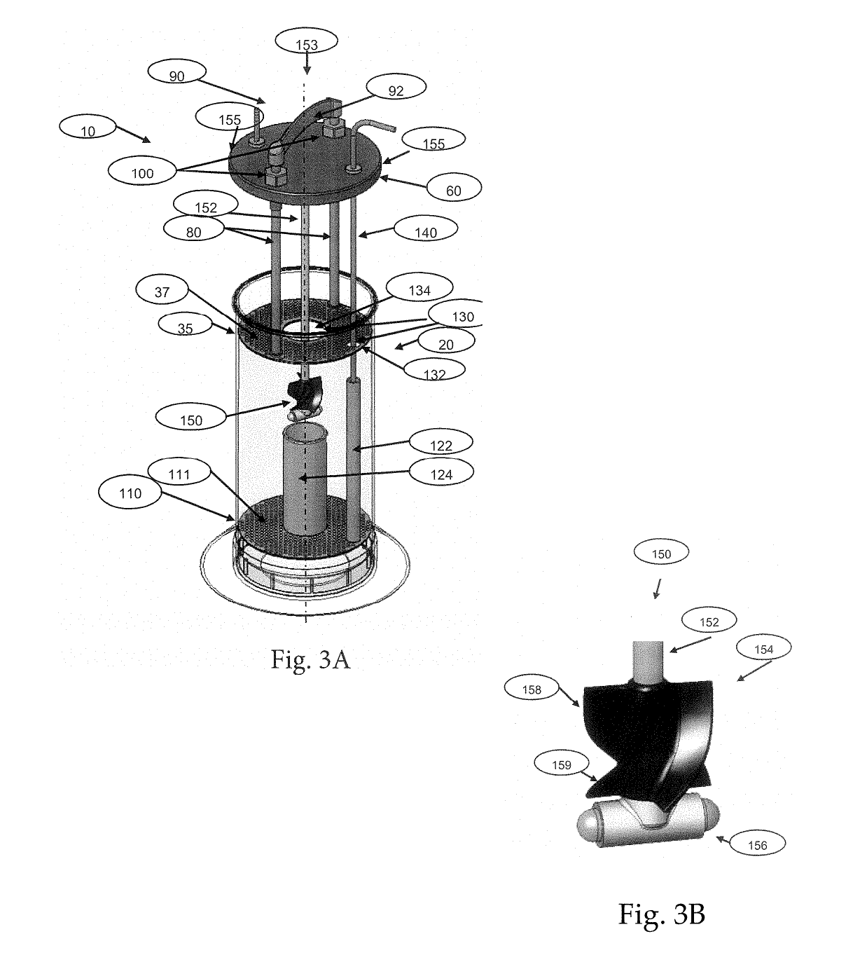

In other embodiments, the fibrous 3D carriers are packed within a middle region 42 (see description below) (which alternatively may be referred to in the art as a “basket”), and the rotary motion is imparted by rotation of the basket. In some embodiments, the basket comprises porous walls, or is bounded by porous walls, which define a radial boundary of the basket that walls within the radial boundary of the basket. In certain embodiments, the porous walls are sufficiently porous to allow free exchange of aqueous solution between the areas within and outside the basket. Alternatively or additionally, the basket is bounded by upper and lower structures (e.g. see component 35 and lower component 110 in the description of FIGS. 2 and 3A, hereinbelow), which may independently be, in some embodiments, porous. In certain embodiments, the 3D carriers are packed sufficiently tightly that their centers of mass do not substantially move relative to the basket walls, during the described rotation of the basket. Typically, the basket also contains the aqueous solution. In more specific embodiments, the basket may be disposed within an outer container, while the aqueous solution is present in the basket and the outer container, and the basket is rotated relative to the outer container. Other embodiments of baskets described herein are bounded only by upper and lower structures and not by walls.

In some embodiments, the basket is rotated in a continuous rotary motion. In more specific embodiments, the rotary motion is between 100-400 rpm; in other embodiments 100-350 rpm; in other embodiments 100-300 rpm; in other embodiments 120-300 rpm; in other embodiments 100-250 rpm; in other embodiments 120-250 rpm; in other embodiments 50-250 rpm in other embodiments 60-250 rpm in other embodiments 80-250 rpm; or in other embodiments 150-250 rpm. Alternatively or in addition, the protruding objects are rotated for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes.

In still other embodiments, the basket is rotated in an oscillating rotary motion. In some embodiments, the protruding objects are subjected to rotational force (relative to a central axis of the chamber) in an oscillating fashion for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes. Alternatively or in addition, the frequency of the oscillation may be once per 1-10 seconds; 1-15 seconds; 1-20 seconds; 1-30 seconds; 1-45 seconds; 1-60 seconds; 2-10 seconds; 2-15 seconds; 2-20 seconds; 2-30 seconds; 2-45 seconds; 2-60 seconds; 3-10 seconds; 3-15 seconds; 3-20 seconds; 3-30 seconds; 3-45 seconds; 3-60 seconds; 5-10 seconds; 5-15 seconds; 5-20 seconds; 5-30 seconds; 5-45 seconds; 5-60 seconds; 10-20 seconds; 10-30 seconds; 10-45 seconds; or 10-60 seconds.

The maximal angular velocity of the described rotary motion (which is, in some embodiments, continuous rotary motion, or is, in other embodiments, oscillating rotary motion) is, in certain embodiments, 3-30, 3-25, 3-20, 3-15, 3-10, 4-30, 4-25, 4-20, 4-15, 4-10, 5-30, 5-25, 5-20, 5-15, 5-10, 6-30, 6-25, 6-20, 6-15, 6-10, 8-30, 8-25, 8-20, 8-15, or 8-10 (rpm).

Each of the aforementioned embodiments of velocity, duration, type, and frequency (in the case of oscillating motion) of the rotary motion may be freely combined.

In certain embodiments, the described agent is removed prior to the subjecting the 3D carriers to a rotary motion. This may be accomplished, in various embodiments, by removing an aqueous solution comprising the agent and replacing it with a solution lacking the agent. In still other embodiments, the carriers are incubated with a washing solution, which is subsequently replaced with another solution lacking the agent, known in the art as a “washing step”.

In other embodiments, the agent is present during the step of subjecting the 3D carriers to a rotary motion. In still other embodiments, the 3D carriers are subjected to a rotary motion in the presence of the agent, then the agent is removed, and the 3D carriers are subjected to an additional rotary motion in the absence of the agent. The two rotary motions in this embodiment may have independently exhibit any of the aforementioned embodiments of velocity, duration, type, and frequency (in the case of oscillating motion), which may be freely combined.

Also provided herein is a method of detaching adherent cells from grooved, rigid, 3D carriers, comprising the steps of: (a) incubating the adherent cells with an agent that disrupts adhesion of the adherent cells to the carrier; and (b) subjecting the 3D carriers to a rotary motion while the 3D carriers are submerged in an aqueous solution. PCT Publication Number WO/2014/037862 to Eytan Abraham et al, published on Mar. 13, 2014, which is incorporated herein by reference in its entirety, describes some embodiments of rigid carriers. These carriers comprise multiple 2D surfaces, wherein the multiple 2D surfaces are configured to support monolayer growth of eukaryotic cells over at least a majority of the 2D surfaces.

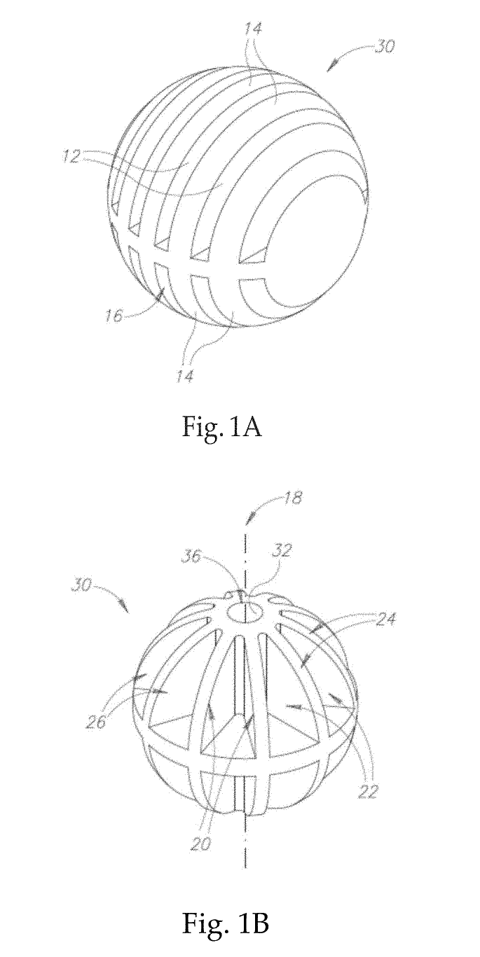

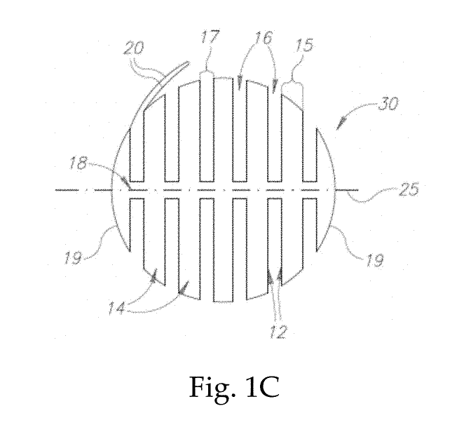

In some embodiments, with reference to FIGS. 1A-B, and as described in WO/2014/037862, published on Mar. 13, 2014, which is incorporated herein by reference in its entirety, grooved carriers 30 are used for proliferation and/or incubation of ASC. In various embodiments, the carriers may be used following a 2D incubation (e.g. on culture plates or dishes), or without a prior 2D incubation.

With reference to FIG. 1A, carriers 30 can include multiple two-dimensional (2D) surfaces 12 extending from an exterior of carrier 30 towards an interior of carrier 30. As shown, the surfaces are formed by a group of ribs 14 that are spaced apart to form openings 16, which may be sized to allow flow of cells and culture medium (not shown) during use. With reference to FIG. 1C, carrier 30 can also include multiple 2D surfaces 12 extending from a central carrier axis 18 of carrier 30 and extending generally perpendicular to ribs 14 that are spaced apart to form openings 16, creating multiple 2D surfaces 12. In other embodiments, openings 16 have a cross-sectional shape that is substantially a semicircle arc (see FIG. 1A). In still other embodiments, the central carrier axis 18 is a plane 25 that bisects the sphere, and openings 16 extend from the surface of the carrier to the proximal surface of the plane. In yet other embodiments, openings 16 extend from the surface 20 of the carrier 30 to the proximal surface of the plane and have a cross-sectional shape that is substantially a semicircle arc. In still other embodiments, carrier 30 is substantially spherical and has a largest diameter of 4-10 millimeter (mm), or between 4-9 mm, 4.5-8.5 mm, 5-8 mm, 5.5-7.5 mm, 6-7 mm, 6.1-6.9 mm, 6.2-6.8 mm, 6.3-6.7 mm, 6.4-6.6 mm, or substantially 6.5 mm. In certain embodiments of the aforementioned carrier, ribs 14 are substantially flat and extend parallel to one another. In more specific embodiments, there are 3-7, 4-6, or 5 parallel ribs (not counting the extreme outer ribs 19), forming 6 openings 16 on each side of plane 25. Alternatively or in addition, the width 15 of ribs 14 and the width 17 of openings 16 are such that the ratio of rib width 15 divided by (rib width 15+opening width 17) is between 0.4-0.8, 0.45-0.75, 0.5-0.7, 0.5-0.8, 0.5-0.75, 0.55-0.65, 0.58-0.62, or substantially 0.6.

In other embodiments, carriers 30 are “3D bodies” as described in WO/2014/037862; the contents of which relating to 3D bodies are incorporated herein by reference.

As mentioned, carrier 30 may have a variety of shapes, including but not limited to spherical, cylindrical, cubical, hyper-rectangular, ellipsoid, and polyhedral and/or irregular polyhedral shapes. In some embodiments, the diameter of the minimal bounding sphere (e.g. the diameter of the carrier, in the case of a spherical shape) of carrier 30 can range from 1-50 mm. In other embodiments, the outer largest dimension can range from 2-20 mm, from 3-15 mm, or from 4-10 mm. In other embodiments, the generic chord length of carriers 30 ranges from 0.5-25 mm, from 1-10 mm, from 1.5-7.5 mm, from 2-5 mm, or from 2.5-4 mm. As known to those skilled in the art, generic chord length is described inter alia in Li et al, Determination of non-spherical particle size distribution from chord length measurements. Part 1: Theoretical analysis. Chemical Engineering Science 60(12): 3251-3265, 2005)

Depending upon the overall size of carrier 30, ribs 14 and openings 16 can be variously sized. For example, ribs 14 can range in thickness from 0.1-2 mm or from 0.2 mm-1 mm. In particular, ribs 14 can be 0.4-0.6 mm, 0.5-0.7 mm, or 0.6-0.8 mm in thickness. Openings 16 can range in width from 0.01-1 mm or from 0.1-0.5 mm. In particular, openings 16 can be 0.25-0.35 mm, 0.35-0.45 mm, or 0.45-0.55 mm in width.

In preferred embodiments, the carriers provide 2D surfaces for attachment and monolayer growth over at least a majority of or all of the surface area of the multiple 2D surfaces 12, 22. Alternatively or in addition, the carriers have a surface area to volume ratio is between 3-1000 cm²/cm³, between 3-500 cm²/cm³, between 3-300 cm²/cm³, between 3-200 cm²/cm³, between 3-100 cm²/cm³, between 3-50 cm²/cm³, between 3-30 cm²/cm³, between 5-20 cm²/cm³, or between 10-15 cm²/cm³.

As shown in FIGS. 1A-B, in various embodiments, carriers 30 may be substantially spherical and have a diameter that forms the carriers' largest dimension. In some embodiments, a diameter of carrier 30 can range from 1-50 mm. In other embodiments, the diameter can range from 2-20 mm, 3-15, mm, or 4-10 mm. With reference to FIG. 1B, depending upon the overall size of carrier 30, ribs 24 and openings 26 can be variously sized. For example, ribs 24 can range in thickness from 0.1-2 mm or from 0.2-1 mm. In particular, ribs 24 can be 0.45-0.55 mm, 0.55-0.65 mm, or 0.65-0.75 mm in thickness. In some embodiments, a minimum width of openings 26 can range from 0.01-1 mm, from 0.05-0.8 mm, or from 0.1-0.5 mm. Specifically, the minimum width of openings 26 can be 0.25-0.35 mm, 0.3.5-0.45 mm, or 0.45-0.55 mm. In other embodiments, the largest cross-sectional dimension of opening 26 can range from 0.1-5 mm, from 0.2-3 mm, or from 0.5-2 mm. More particularly, opening 26 can have a largest cross-sectional dimension of 0.7.5-0.85 mm, 0.95-1.05 mm, or 1.15-0.25 mm. Further, carrier 30 includes an opening 36 extending through the carrier's center and forming additional surfaces 32, which can support monolayer growth of eukaryotic cells.

In the embodiment shown in FIG. 1A, ribs 14 are substantially flat and extend parallel to one another. In other embodiments, the ribs are in other configurations. For example, FIG. 1B illustrates carrier 30 having multiple two-dimensional surfaces 22 formed by ribs 24 in a different configuration. In particular, ribs 24 are shaped to form openings 26 that are spaced around the circumference of carrier 30, whereby openings 26 can be generally wedge shaped. Ribs 24 can extend generally radially from a central carrier axis 18 of carrier 30 to a peripheral surface of carrier 30. Carrier 30 can also include one or more lateral planes extending from the central carrier axis 18 of carrier 30 and extending generally perpendicular to ribs 24, as depicted in FIG. 1C, which is a cross-sectional view of certain embodiments of the carrier 30 of FIG. 1A.

In still other embodiments, the material forming the multiple 2D surfaces comprises at least one polymer. In more specific embodiments, the polymer is selected from a polyamide, a polycarbonate, a polysulfone, a polyester, a polyacetal, and polyvinyl chloride.

The material used to produce the described carriers can include, in various embodiments, metals (e.g. titanium), metal oxides (e.g., titanium oxide films), glass, borosilicate, carbon fibers, ceramics, biodegradable materials (e.g. collagen, gelatin, PEG, hydrogels), and or polymers. Suitable polymers may include polyamides, such as GRILAMID® TR 55 (EMS-Grivory, Sumter, S.C.); polycarbonates such as LEXAN® (Sabic, Pittsfield, Mass.) and Macrolon® (Bayer); polysulfones such as RADEL® PPSU (Solvay) and UDEL® PSU (Solvay); polyesters such as TRITAN® (Polyone) and PBT® HX312C; polyacetals such as CELON® (Ticana), and polyvinyl chloride. In certain embodiments, the described carriers are composed of a non-porous material, or, if pores are present, they are no larger than 20 microns, in other embodiments 10 microns, in other embodiments 5 microns, in other embodiments 3 microns, in other embodiments 2 microns, or in other embodiments 1 micron.

In more specific embodiments, cell-culture carriers are formed of injection-molded surface treatment of LEXAN® or GRILAMID®, with a smooth surface texture, using growth medium proteins and/or polylysine on LEXAN® or GRILAMID® carriers; cell-culture carriers formed of injection-molded GRILAMID® with a rough surface that was preincubated with growth medium proteins. In other embodiments, untreated LEXAN® or GRILAMID® surfaces are utilized.

In other embodiments, at least part of the carriers may be formed using a polystyrene polymer. The polystyrene may be further modified using corona discharge, gas-plasma (roller bottles and culture tubes), or other similar processes. These processes can generate highly energetic oxygen ions which graft onto the surface polystyrene chains so that the surface becomes hydrophilic and negatively charged when medium is added. Furthermore, any of the carriers may be produced at least in part from combinations of materials. Materials of the carriers can be further coated or treated to support cell attachment. Such coating and/or pretreatment may include use of collagen I, collagen IV, gelatin, poly-d-lysine, fibronectin, laminin, amine, and carboxyl.

In various embodiments, the described carriers are coated with one or more coatings. Suitable coatings may, in some embodiments, be selected to control cell attachment or parameters of cell biology. Suitable coatings may include, for example, peptides, proteins, carbohydrates, nucleic acid, lipids, polysaccharides, glycosaminoglycans, proteoglycans, hormones, extracellular matrix molecules, cell adhesion molecules, natural polymers, enzymes, antibodies, antigens, polynucleotides, growth factors, synthetic polymers, polylysine, drugs and/or other molecules or combinations or fragments of these.

Furthermore, in various embodiments, the surfaces of the carriers described herein may be treated or otherwise altered to control cell attachment and or other biologic properties. Options for treating the surfaces including chemical treatment, plasma treatment, and/or corona treatment. Further, in various embodiments, the materials may be treated to introduce functional groups into or onto the material, including groups containing hydrocarbons, oxygen, and/or nitrogen. In addition, in various embodiments, the material may be produced or altered to have a texture to facilitate settling of cells or control other cell properties. For example, in some embodiments, the materials used to produce the cell-culture carriers have a roughness on a nanometer or micrometer scale that facilitates settling of cells and/or controls other cell properties.

In certain embodiments, the rigid carriers and the aqueous solution are disposed within a chamber, and the rotary motion is imparted by protruding objects projecting radially from a central axis of the chamber. In more specific embodiments, the described chamber is cylindrical or a similar shape, e.g. an irregular cylinder, whose cross-sectional area is not constant along the axis of the cylinder. In other embodiments, the cylinder is an elliptic cylinder, parabolic cylinder, or hyperbolic cylinder, namely a cylinder whose cross section is an ellipse, parabola, or hyperbola, respectively. In still other embodiments, the cylinder is an oblique cylinder. The described protruding objects, in some embodiments, extend radially from an axial element that is configured to rotate and is aligned with the central axis. In certain embodiments, axial element is cylindrical or is essentially cylindrical, having a small diameter than the diameter of the chamber. “Axial” in this context refers to a line connecting the centers of the bases of a cylinder or a similar shape.

The described protruding objects, are in certain embodiments, spoke-like projections. In other embodiments, the protruding objects may be rod-shaped. Alternatively or in addition, there may be 10-50, 10-40, 10-30, 15-50, 15-40, 15-30, 15-25, 17-23, 20-30, or 22-28 spokes extending radially from a central axis. In still other embodiments, the protruding objects and axial element may form a helix conformation.

In still other embodiments, the length of the protruding objects (perpendicular to the chamber axis) is 50-90%, 50-95%, 50-80%, 40-95%, 40-90%, 40-80%, 40-70%, or 50-70% of the inner cross-sectional radius of the chamber, wherein the measurement extends from the external surface of the axial element to the inner surface of the chamber wall. Thus, the protruding objects extend the indicated percentage of the distance from the external surface of the axial element to the inner surface of the chamber wall.

Alternatively or in addition, the radius of the protruding objects (parallel to the chamber axis) is 0.5-5%, 0.5-4%, 0.5-3%, 0.5-2%, 0.5-1.5%, 0.4-5%, 0.4-4%, 0.4-3%, 0.4-2%, 0.4-1.5%, 0.3-5%, 0.3-4%, 0.3-3%, 0.3-2%, or 0.3-1.5% of the chamber axis.

In some embodiments, the protruding objects are rotated in a continuous rotary motion. In more specific embodiments, the rotary motion is between 100-400 rpm; in other embodiments 100-350 rpm; in other embodiments 100-300 rpm; in other embodiments 120-300 rpm; in other embodiments 100-250 rpm; in other embodiments 120-250 rpm; in other embodiments 50-250 rpm in other embodiments 60-250 rpm in other embodiments 80-250 rpm; or in other embodiments 150-250 rpm. Alternatively or in addition, the protruding objects are rotated for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes.

In still other embodiments, the protruding objects are subjected to rotational force (relative to a central axis of the chamber) in an oscillating fashion. In some embodiments, the protruding objects are moved in an oscillating rotary motion for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes. Alternatively or in addition, the frequency of the oscillation may be once per 1-10 seconds; 1-15 seconds; 1-20 seconds; 1-30 seconds; 1-45 seconds; 1-60 seconds; 2-10 seconds; 2-15 seconds; 2-20 seconds; 2-30 seconds; 2-45 seconds; 2-60 seconds; 3-10 seconds; 3-15 seconds; 3-20 seconds; 3-30 seconds; 3-45 seconds; 3-60 seconds; 5-10 seconds; 5-15 seconds; 5-20 seconds; 5-30 seconds; 5-45 seconds; 5-60 seconds; 10-20 seconds; 10-30 seconds; 10-45 seconds; or 10-60 seconds.

The maximal angular velocity of the described rotary motion (which is, in some embodiments, continuous rotary motion, or is, in other embodiments, oscillating rotary motion) is, in certain embodiments, 3-30, 3-25, 3-20, 3-15, 3-10, 4-30, 4-25, 4-20, 4-15, 4-10, 5-30, 5-25, 5-20, 5-15, 5-10, 6-30, 6-25, 6-20, 6-15, 6-10, 8-30, 8-25, 8-20, 8-15, or 8-10 rotations per minute (rpm).

Each of the aforementioned embodiments of the number, geometry, and arrangement of the protruding objects and the velocity, duration, type, and frequency (in the case of oscillating motion) of the rotary motion may be freely combined.

In other embodiments, the rigid 3D carriers are packed within a middle region 42 (see description below) (which alternatively may be referred to in the art as a “basket”), and the rotary motion is imparted by rotation of the basket. In still other embodiments, the basket comprises porous walls, or is bounded by porous walls, which define a radial boundary of the basket that walls within the radial boundary of the basket. In certain embodiments, the porous walls are sufficiently porous to allow free exchange of aqueous solution between the areas within and outside the basket. Alternatively or additionally, the basket is bounded by upper and lower structures (e.g. see component 35 and lower component 110 in the description of FIGS. 2 and 3A, hereinbelow), which may independently be, in some embodiments, porous. In certain embodiments, the 3D carriers are packed sufficiently tightly that their centers of mass do not substantially move relative to the basket walls, during the described rotation of the basket. Typically, the basket also contains the aqueous solution. In more specific embodiments, the basket may be disposed within an outer container, while the aqueous solution is present in the basket and the outer container, and the basket is rotated relative to the outer container. Some embodiments of baskets described herein are bounded only by upper and lower structures and not by walls.

In some embodiments, the basket is rotated in a continuous rotary motion. In more specific embodiments, the rotary motion is between 100-400 rpm; in other embodiments 100-350 rpm; in other embodiments 100-300 rpm; in other embodiments 120-300 rpm; in other embodiments 100-250 rpm; in other embodiments 120-250 rpm; in other embodiments 50-250 rpm in other embodiments 60-250 rpm in other embodiments 80-250 rpm; or in other embodiments 150-250 rpm. Alternatively or in addition, the protruding objects are rotated for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes.

In still other embodiments, the basket is rotated in an oscillating rotary motion. In some embodiments, the protruding objects are subjected to rotational force (relative to a central axis of the chamber) in an oscillating fashion, for a time between 0.5-15, 0.6-15, 0.8-15, 1-15, 2-15, 3-15, 4-15, 5-15, 0.5-20, 0.6-20, 0.8-20, 1-20, 2-20, 3-20, 4-20, 5-20, 0.5-30, 0.6-30, 0.8-30, 1-30, 2-30, 3-30, 4-30, or 5-30 minutes. Alternatively or in addition, the frequency of the oscillation may be once per 1-10 seconds; 1-15 seconds; 1-20 seconds; 1-30 seconds; 1-45 seconds; 1-60 seconds; 2-10 seconds; 2-15 seconds; 2-20 seconds; 2-30 seconds; 2-45 seconds; 2-60 seconds; 3-10 seconds; 3-15 seconds; 3-20 seconds; 3-30 seconds; 3-45 seconds; 3-60 seconds; 5-10 seconds; 5-15 seconds; 5-20 seconds; 5-30 seconds; 5-45 seconds; 5-60 seconds; 10-20 seconds; 10-30 seconds; 10-45 seconds; or 10-60 seconds.

The maximal angular velocity of the described rotary motion (which is, in some embodiments, continuous rotary motion, or is, in other embodiments, oscillating rotary motion) is, in certain embodiments, 3-30, 3-25, 3-20, 3-15, 3-10, 4-30, 4-25, 4-20, 4-15, 4-10, 5-30, 5-25, 5-20, 5-15, 5-10, 6-30, 6-25, 6-20, 6-15, 6-10, 8-30, 8-25, 8-20, 8-15, or 8-10 (rpm).

Each of the aforementioned embodiments of velocity, duration, type, and frequency (in the case of oscillating motion) of the rotary motion may be freely combined.

In certain embodiments, the described agent is removed prior to the subjecting the 3D carriers to a rotary motion. This may be accomplished, in various embodiments, by removing an aqueous solution comprising the agent and replacing it with a solution lacking the agent. In still other embodiments, the carriers are incubated with a washing solution, which is subsequently replaced with another solution lacking the agent, known in the art as a “washing step”.

In other embodiments, the agent is present during the step of subjecting the 3D carriers to a rotary motion. In still other embodiments, the 3D carriers are subjected to a rotary motion in the presence of the agent, then the agent is removed, and the 3D carriers are subjected to an additional rotary motion in the absence of the agent. The two rotary motions in this embodiment may have independently exhibit any of the aforementioned embodiments of velocity, duration, type, and frequency (in the case of oscillating motion), which may be freely combined.

The aforementioned agent that disrupts adhesion of the adherent cells to the carrier, is, in some embodiments, a protease. In certain embodiments, the protease is present in an aqueous solution, which may, in further embodiments, comprise a chelator of divalent cations, for example a chelator of calcium and/or magnesium, a non-limiting example of which is ethylenediaminetetraacetic acid (EDTA). Non-limiting examples of suitable proteases are Trypsin and other enzymes with similar activity, non-limiting examples are TrypLE™, a fungal trypsin-like protease. Such enzymes are in some embodiments used in combination with another enzyme, for example Collagenase, non-limiting examples of which are Collagenase Types I, II, III, and IV (which are available commercially from Life Technologies), and other enzymes with similar activity, non-limiting examples of which are Dispase I and Dispase II, which are available commercially from Sigma-Aldrich. In various embodiments, incubation with the described agent can be for 1-30, 2-30, 3-30, 4-30, 5-30, 6-30, 8-30, 10-30, 1-20, 2-20, 3-20, 4-20, 5-20, 6-20, 8-20, 10-20, 1-10, 2-10, 3-10, 4-10, 5-10, 6-10, 8-10, 10-20, 11-19, 12-18, 13-17, 14-16, 12-20, or 15-20 minutes;

In other embodiments, the aforementioned agent is an agent that disrupts adhesion of the adherent cells to an extracellular matrix (ECM). Alternatively or in addition, the agent disrupts adhesion of the adherent cells to neighboring cells; and/or disrupts focal adhesions. In still other embodiments, the agent is an agent that cleaves peptide bonds.

In various embodiments, the systems of the present disclosure can be used to harvest a variety of different eukaryotic cell types. For example, the systems can be suitable for growth of stem cells, anchorage-dependent cells, mesenchymal cells, and adherent cells. As used herein the phrase “adherent cells” refers to cells that are capable of attaching to an attachment substrate and expanding or proliferating on the substrate. In some embodiments, the cells are anchorage dependent, i.e., require attachment to a surface in order to proliferate grow in vitro. Suitable adherent cells can include adherent stromal cells (ASC). In various embodiments, the ASC are obtained from, e.g., a source selected from bone marrow, adipose tissue, placenta, cord blood, and peripheral blood. Alternatively or in addition, in various embodiments, the ASC are or are not be capable of differentiating into different types of cells (e.g. reticular endothelial cells, fibroblasts, adipocytes, osteogenic precursor cells), depending upon influences from bioactive factors.

In other embodiments, the described ASC are placenta-derived. Except where indicated otherwise herein, the terms “placenta”, “placental tissue”, and the like refer to any portion of the placenta. Placenta-derived adherent cells may be obtained, in various embodiments, from either fetal or, in other embodiments, maternal regions of the placenta, or in other embodiments, from both regions. More specific embodiments of maternal sources are the decidua basalis and the decidua parietalis. More specific embodiments of fetal sources are the amnion, the chorion, and the villi. In certain embodiments, tissue specimens are washed in a physiological buffer [e.g., phosphate-buffered saline (PBS) or Hank's buffer]. In certain embodiments, the placental tissue from which cells are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. More specific embodiments of decidua are decidua basalis, decidua capsularis, and decidua parietalis.

In still other embodiments, the cells are a placental cell population that is a mixture of fetal-derived placental ASC (also referred to herein as “fetal ASC” or “fetal cells”) and maternal-derived placental ASC (also referred to herein as “maternal ASC” or “maternal cells”), where a majority of the cells are maternal cells. In more specific embodiments, the mixture contains at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, at least 99.9%, at least 99.92%, at least 99.95%, at least 99.96%, at least 99.97%, at least 99.98%, or at least 99.99% maternal cells, or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9%, 99-99.9%, 99.2-99.9%, 99.5-99.9%, 99.6-99.9%, 99.7-99.9%, or 99.8-99.9% maternal cells.

In other embodiments, the cells are a placental cell population that does not contain a detectable amount of maternal cells and is thus entirely fetal cells. A detectable amount refers to an amount of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the preparation is a placental cell population that is a mixture of fetal and maternal cells, where a majority of the cells are fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, 99% or 100% of the cells are fetal cells. Expression of CD200, as measured by flow cytometry, using an isotype control to define negative expression, can be used as a marker of fetal cells under some conditions. In yet other embodiments, at least 60%, at least 65%, at least 70%, at least 75%, at least 80%, at least 85%, at least 90%, at least 92%, at least 95%, at least 96%, at least 97%, at least 98%, at least 99%, at least 99.5%, at least 99.7%, or at least 99.9% of the described cells are fetal cells.

Alternatively or in addition, the cells are mesenchymal-like adherent stromal cells (ASC), which exhibit a marker pattern similar to mesenchymal stromal cells, but do not differentiate into osteocytes, under conditions where “classical” mesenchymal stem cells (MSC) would differentiate into osteocytes. In other embodiments, the cells exhibit a marker pattern similar to MSC, but do not differentiate into adipocytes, under conditions where MSC would differentiate into adipocytes. In still other embodiments, the cells exhibit a marker pattern similar to MSC, but do not differentiate into either osteocytes or adipocytes, under conditions where mesenchymal stem cells would differentiate into osteocytes or adipocytes, respectively. The MSC used for comparison in these assays are, in some embodiments, MSC that have been harvested from bone marrow (BM) and cultured under 2D conditions. In other embodiments, the MSC used for comparison have been harvested from BM and cultured under 2D conditions, followed by 3D conditions. In more particular embodiments, the mesenchymal-like ASC are maternal cells. In alternative embodiments, the mesenchymal-like ASC are fetal cells.

Alternatively or additionally, the ASC may express a marker or a collection of markers (e.g. surface marker) characteristic of MSC or mesenchymal-like stromal cells. In some embodiments, the ASC express some or all of the following markers: CD105 (UniProtKB Accession No. P17813), CD29 (UniProtKB Accession No. P05556), CD44 (UniProtKB Accession No. P16070), CD73 (UniProtKB Accession No. P21589), and CD90 (UniProtKB Accession No. P04216). In some embodiments, the ASC do not express some or all of the following markers: CD3 (e.g. UniProtKB Accession Nos. P09693 [gamma chain] P04234 [delta chain], P07766 [epsilon chain], and P20963 [zeta chain]), CD4 (UniProtKB Accession No. P01730), CD11b (UniProtKB Accession No. P11215), CD14 (UniProtKB Accession No. P08571), CD19 (UniProtKB Accession No. P15391), and/or CD34 (UniProtKB Accession No. P28906). In more specific embodiments, the ASC also lack expression of CD5 (UniProtKB Accession No. P06127), CD20 (UniProtKB Accession No. P11836), CD45 (UniProtKB Accession No. P08575), CD79-alpha (UniProtKB Accession No. B5QTD1), CD80 (UniProtKB Accession No. P33681), and/or HLA-DR (e.g. UniProtKB Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In certain embodiments, over 90% of the ASC are positive for CD29, CD90, and CD54. In other embodiments, over 85% of the described cells are positive for CD29, CD73, CD90, and CD105. In yet other embodiments, less than 3% of the described cells are positive for CD14, CD19, CD31, CD34, CD39, CD45RA (an isotype of CD45), HLA-DR, Glycophorin A, and CD200; less than 6% of the cells are positive for GlyA; and less than 20% of the cells are positive for SSEA4. In more specific embodiments, over 90% of the described cells are positive for CD29, CD90, and CD54; and over 85% of the cells are positive for CD73 and CD105. In still other embodiments, over 90% of the described cells are positive for CD29, CD90, and CD54; over 85% of the cells are positive for CD73 and CD105; less than 6% of the cells are positive for CD14, CD19, CD31, CD34, CD39, CD45RA, HLA-DR, GlyA, CD200, and GlyA; and less than 20% of the cells are positive for SSEA4. The aforementioned, non-limiting marker expression patterns were found in certain maternal placental cell populations that were expanded on 3D substrates.

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

In still other embodiments, the majority, in other embodiments over 60%, over 70%, over 80%, or over 90% of the expanded cells express CD29, CD73, CD90, and CD105. In yet other embodiments, less than 20%, 15%, or 10% of the described cells express CD3, CD4, CD34, CD39, and CD106. In yet other embodiments, less than 20%, 15%, or 10% of the described cells highly express CD56. In various embodiments, the cell population may be less than 50%, less than 40%, less than 30%, less than 20%, or less than 10%, or less than 5% positive for CD200. In other embodiments, the cell population is more than 50%, more than 60%, more than 70%, more than 80%, more than 90%, more than 95%, more than 97%, more than 98%, more than 99%, or more than 99.5% positive for CD200. In certain embodiments, more than 50% of the cells express, or in other embodiments highly express, CD141 (thrombomodulin; UniProt Accession No. P07204), or in other embodiments SSEA4 (stage-specific embryonic antigen 4, an epitope of ganglioside GL-7 (IV3 NeuAc 2→3 GalGB4); Kannagi R et al), or in other embodiments both markers. Alternatively or in addition, more than 50% of the cells express HLA-A2 (UniProt Accession No. P01892). The aforementioned, non-limiting marker expression patterns were found in certain fetally-derived placental cell populations that were expanded on 3D substrates. The Uniprot Accession Nos. mentioned in the paragraph were accessed on accessed on Feb. 8, 2017.

In other embodiments, each of CD73, CD29, and CD105 is expressed by more than 90% of the ASC; and the cells do not differentiate into adipocytes, under conditions where mesenchymal stem cells would differentiate into adipocytes. In some embodiments, as provided herein, the conditions are incubation of adipogenesis induction medium, for example a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days. In yet other embodiments, each of CD34, CD45, CD19, CD14 and HLA-DR is expressed by less than 3% of the cells; and the cells do not differentiate into adipocytes, after incubation under the aforementioned conditions. In other embodiments, each of CD73, CD29, and CD105 is expressed by more than 90% of the cells, each of CD34, CD45, CD19, CD14 and HLA-DR is expressed by less than 3% of the cells; and the cells do not differentiate into adipocytes, after incubation under the aforementioned conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days. The aforementioned solutions will typically contain cell culture medium such as DMEM+10% serum or the like, as will be appreciated by those skilled in the art. The aforementioned, non-limiting phenotypes and marker expression patterns were found in certain maternal placental cell populations that were expanded on 3D substrates. These experiments were performed as described in Example 2 of WO 2016/098061, which is incorporated herein by reference.

FIG. 2 is a perspective view of a system 10 for cell growth and harvesting, according to certain embodiments. Although system 10 is ordinarily kept closed, it is generally depicted opened in the Figures herein in order to show the inner components. As shown, system 10 includes a vessel 20 configured to receive a plurality of carriers 30 (see FIGS. 3A-B and description hereinbelow), which may be located within a chamber 40 of vessel 20. For simplicity, only 3 carriers are shown; a much larger number will typically be present.

System 10 can also include a component 35, which serves to confine carriers by preventing them from moving further towards upper plate 60. In certain embodiments, component 35 may be configured to allow packing and unpacking of carriers 30 within vessel 20. As shown in FIGS. 2 and 3A, component 35 may be located inside said chamber 40 of vessel 20. Component 35 may separate chamber 40 into one or more regions. For example, component 35 may separate chamber 40 into a middle region 42 and a second region 44. In operation, middle region 42 may be filled with carriers 30 (not shown). Component 35 may then be moved to apply a compressive force to carriers 30 to pack them into a specific volume of middle region 42. In this packed configuration, carriers 30 may remain stationary within middle region 42. Medium 145 in second region 44 may be stirred and may move through middle region 42, allowing growth of cells on carriers 30 contained within middle region 42. In addition, chamber 40 may include a third region 46. Second and third regions 44, 46 may be located on two or more sides of middle region 42 to provide buffer regions where medium 145 may be stirred to provide flow of medium 145 across carriers 30 located within middle region 42. Accordingly, component 35 (also referred to as the “second perforated structure”) may be porous and may have upper pores 37 of sufficient size to permit flow of medium 145 across component 35, yet retain carriers within middle region 42. Component 35 could be formed of any suitable material, such as, for example, a polymer, a metal alloy, or a combination of various materials. In certain embodiments, component 35 may be configured to provide a plunger-like action whereby movement of component 35 compresses carriers 30 located within chamber 40. In other embodiments, middle region 42 may be subdivided into multiple compartments by adding one or more additional component 35 (not depicted)

In some embodiments, component 35 may be moveably coupled to vessel 20. As shown in FIGS. 2 and 3A, component 35 may be moved vertically relative to vessel 20. In other embodiments, component 35 may be moved horizontally, may expand or contract, or may be moved in some other way to restrict the movement of carriers 30 within vessel 20.

As shown in FIGS. 2 and 3A, system 10 can include an upper cover or plate 60 configured to couple to vessel 20. In some embodiments, upper cover or plate 60 can be configured to seal chamber 40, which is configured to receive a range of carriers. Upper cover or plate 60 can also include one or more ports 70. System 10 can include tubing, sensors, mechanical members, impeller shafts and other devices requiring access to chamber 40.

For example, upper plate 60 can include one or more ports 70 configured to receive one or more support members 80 coupled to component 35. Ports 70 are preferably sealed such as to prevent introduction of bacteria or other biological contaminants. As illustrated in the embodiments shown in FIGS. 2 and 3A, two support members 80 can be fixedly coupled to component 35, and may extend upwards from component 35 and through two ports 70 of upper cover or plate 60. And while shown as separate, component 35 and support members 80 could be formed as a single-piece or monolithic device. Component 35 and support members 80 could be formed from plastic, metal, glass, or other suitable material.

Component 35 and/or support members 80 could also be coupled to a handle 90. Handle 90 can include a grip 92 configured to allow an operator to move component 35 from a first position to a second position as described above. As shown in FIG. 2, handle 90 may be used to raise and lower component 35 to pack and unpack carriers 30.

System 10 can also include one or more locking elements 100 configured to lock component 35 in one or more positions. For example, locking element 100 could lock component 35 in at least one of the first position and the second position relative to vessel 20. Locking element 100 is depicted as a bolt, but could include any similar device that engages upper plate 60 and/or support member 80. The mechanism could for example utilize a thread (not visible). Locking element 100 could also include a latch, cleat, friction fit, button, gear, motor, or other device (not depicted) configured to lock component 35, support member 80, and/or handle 90 in one or more positions relative to vessel 20.

System 10 may also include a lower component 110 configured to support carriers within vessel 20. As shown in FIGS. 2 and 3A, lower component 110 can be located at a distance from a lower surface of vessel 20. In particular, lower component 110 can be shaped and sized for locating within chamber 40 such that third region 46 includes a volume of medium 145. Similar to component 35 described above, lower component 110 (also referred to as the “first perforated structure”) can be porous with lower pores 111 of sufficient size to permit flow of medium 145 between middle region 42 and third region 46 while maintaining carriers within middle region 42. In certain embodiments, carriers 30 are located in middle region 42 between component 35 and lower component 110.

In some embodiments, component 35 and/or lower component 110 may also include one or more apertures 130 configured to receive one or more conduits 122,124. As shown in FIG. 3A, multiple conduits 122,124 can extend generally between component 35 and lower component 110 via apertures 130. Conduits 122,124 can provide direct fluid passage between second region 44 and third region 46. For example, a first conduit 122 and a second conduit 124 can extend upwards from lower member 110 towards component 35. Component 35 may include one or more apertures 130 sized and located to receive associated conduits 122,124. In particular, component 35 can include a first aperture 132 associated with first conduit 122 and a second aperture 134 associated with second conduit 124. As shown in FIG. 3A, first conduit 122 and first aperture 132 can be located, shaped, and sized to receive a line 140. Line 140 may provide a direct passageway for transport of fluid to or from third region 46 and through upper plate 60.

As shown in FIG. 3A, shaft 152, second conduit 124, and second aperture 134 may be located on a central longitudinal axis 153 of vessel 20. Line 140, first conduit 122, and first aperture 132 may be located about a periphery 155 of chamber 40, off the central longitudinal axis 153 of vessel 20.

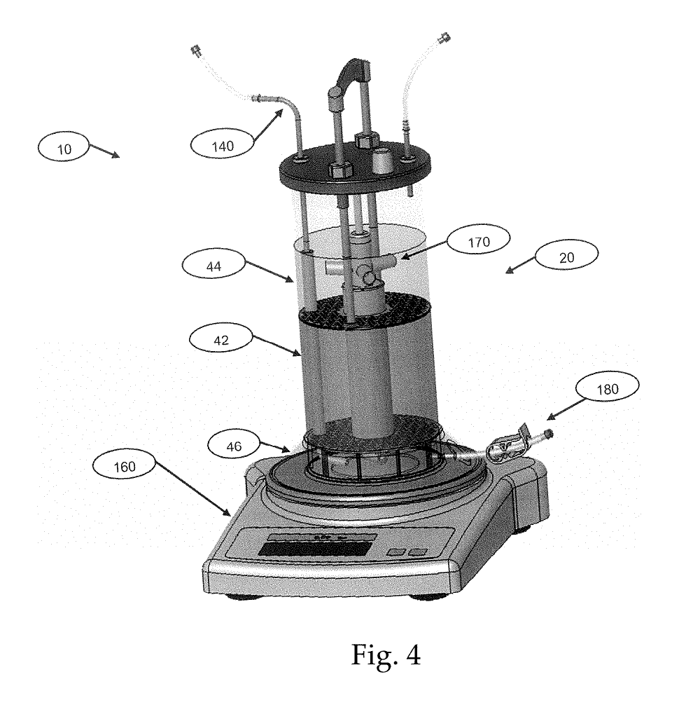

Second conduit 124 and second aperture 134 may also be located, shaped, and sized to receive a bladed impeller 150. As shown in FIGS. 3A-B, bladed impeller 150 can include a shaft 152 optionally coupled to upper plate 60, a blade 154 configured to move fluid when rotated, and/or a magnetic element 156 configured to magnetically couple to a stirring device 160 (FIG. 4). In certain embodiments (FIG. 3B), bladed impeller 150 comprises 2-5 blades 154 that extend substantially radially from shaft 152 throughout their length. The plane of the distal end 158 of the blade 154 relative to magnetic element 156 is substantially parallel to the axis (90 degrees [deg.] slope relative to magnetic element 156), and the slope of the plane gradually decreases from 90 degrees (deg.) to reach between 30-60° at the proximal end 159 of the blade 154 relative to magnetic element 156. In certain embodiments, bladed impeller 150 may rotate to move medium 145 through middle region 42 and over cells growing on carriers 30. Shaft 152 can be fixedly coupled to upper plate 60 and rotationally coupled to blade 154 and/or magnetic element 156. Alternatively, shaft 152 can be fixedly coupled to blade 154, and/or magnetic element 156, and/or can be rotationally coupled to upper plate 60. In other embodiments, shaft 152 could be coupled to a motor (not shown) or other device configured to rotate shaft 52 and blade 154. Such an embodiment would not require magnetic element 156.

It is contemplated that various features described above may be provided on different devices and that different configurations of devices are possible. For example, conduits 122,124 may be coupled to component 35, upper plate 60, or another part of system 10. Likewise apertures may be provided in lower component 110, vessel 20, or another part of system 10. In some embodiments, an upper impeller 170 may be provided in second region 44 of chamber 40 to aid circulation of medium 145 throughout chamber 40. Additional probes, lines, and other devices (not depicted) may be provided within chamber 40 and different regions 42, 44, and 46. Accordingly, component 35 and lower component 110 may be configured to operate with these additional devices.

Other examples of possible alternate embodiments include providing one or more impellers 150 within one or more regions 42, 44, 46 of chamber 40. A lower line 180 may (FIG. 4) or may not (FIG. 3A) be fluidly coupled directly to third region 46 through a wall of vessel 20. And in some embodiments, system 10 may not require lower component 110 or third region 46. Sufficient flow of medium 145 may be achieved using lines suitably positioned about the lower part of chamber 40. For example, additional lines (not shown) may extend down from upper plate 60 about a periphery 155 of chamber 40 with openings into middle region 42. Single or multiple inlet and/or outlet lines 140 and/or conduits 122,124, containing one or more openings, could provide middle region 42 with sufficient flow of medium 145 to provide adequate incubation conditions. In other embodiments, conduit 124 may be removed and/or a suitably configured impeller, for example a bladed impeller 150, may be used to move medium 145 through middle region 42. In yet another embodiment, a packed bed of carriers 30 may be moved as a single entity within the medium 145.

As shown in FIG. 4 and described above, system 10 can include stirring device 160. Stirring device 160 can be set manually or programmed to automatically specific rates of rotation to one or more impellers 150, 170.

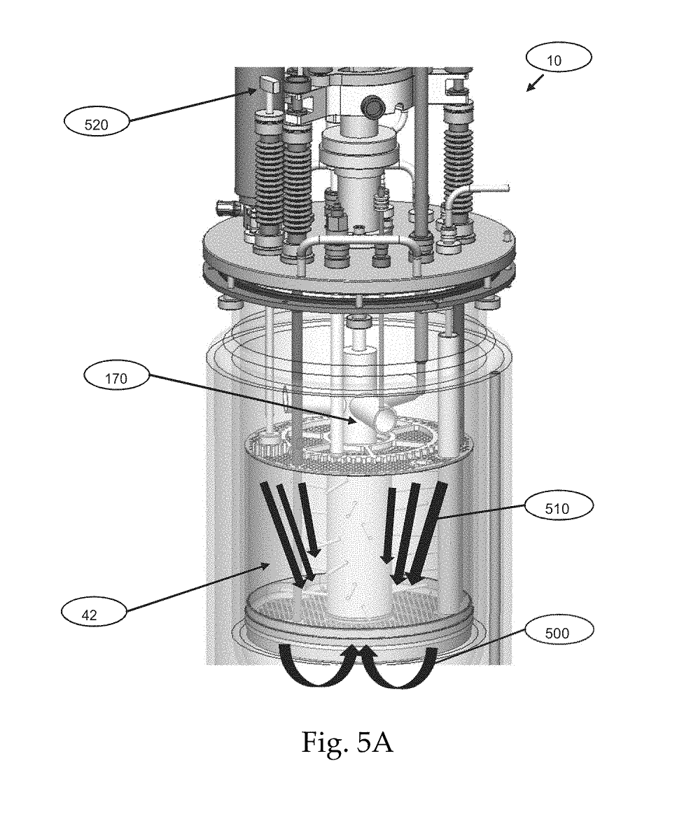

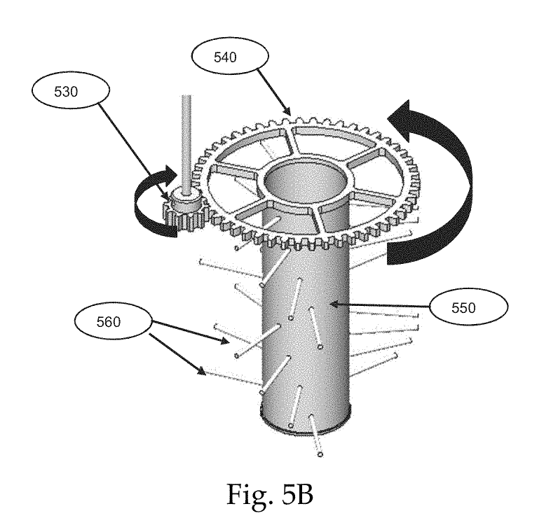

In other embodiments of system 10, with reference to FIG. 5A, vessel 20 comprises a rotating cylinder 550, typically along the central axis of vessel 20. Spokes 560 extend radially from rotating cylinder 550 inside middle region 42, imparting motion to carriers 30 when rotating cylinder 550 is rotated. In certain embodiments, with reference to FIG. 5B, rotating cylinder 550 may be operably connected with an external component capable of transmitting applied torque, such as handle 520, which may optionally be either manually rotatable or connected with a motor (not depicted), via shaft 152. In some embodiments, the operative connection comprises a gear mechanism, including small gear 530 and large gear 540. Rotating cylinder 550 can be used to impart a rotary motion to spokes 560 at the time of harvesting, thus moving carriers 30 in a revolving fashion around middle region 42 and in some cases imparting a degree of spinning motion to carriers 30. Rotating cylinder 550 may rotate either in continuous rotary motion, or in other embodiments in a partial rotary motion, for example an oscillating partial rotary motion.

In still other embodiments (not depicted), a rotating cylinder is operably connected to a basket that holds the described fibrous 3D carriers or rigid 3D carriers, enabling rotation of the basket relative to the outer chamber wall.

Optionally, cell-lift impeller 170 is also present, creating a vacuum pull 500 below, leading to downward fluid flow 510 in middle region 42. Cell-lift impeller 170 preferably rotates around the same axis as rotating cylinder 550, but is not operably connected with rotating cylinder 550. The term “cell-lift” impeller may refer to an impeller including a vertical tube, whose rotating motion creates a low-differential pressure the base of the tube.

In certain embodiments, 3D culturing is performed in a bioreactor designed for containing 3D carriers. In some embodiments, the 3D bioreactor comprises a container for holding medium and a 3-dimensional attachment (carrier) substrate disposed therein, and a control apparatus, for controlling pH, temperature, and oxygen levels and optionally other parameters. Alternatively or in addition, the bioreactor contains ports for the inflow and outflow of fresh medium and gases. Except where indicated otherwise, the term “bioreactor” excludes decellularized organs and tissues derived from a living being.

Examples of bioreactors include, but are not limited to, a continuous stirred tank bioreactor, a CelliGen Plus® bioreactor system (New Brunswick Scientific (NBS) and a BIOFLO 310 bioreactor system (New Brunswick Scientific (NBS).

In certain embodiments, a 3D bioreactor is capable of 3D expansion of adherent stromal cells under controlled conditions (e.g. pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, N.J.). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference. A “stationary-bed bioreactor” refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during the standard agitation rate of the bioreactor. In some embodiments, multiple carriers are loosely packed, for example forming a loose packed bed, which is submerged in a nutrient medium. In certain embodiments, the carriers, although remaining substantially in place in the absence of exertion of rotational force on them, can be readily moved by rotation of the described protruding objects. In other embodiments, the substrate may be lifted at unusually fast agitation rates, for example greater than 200 rpm.

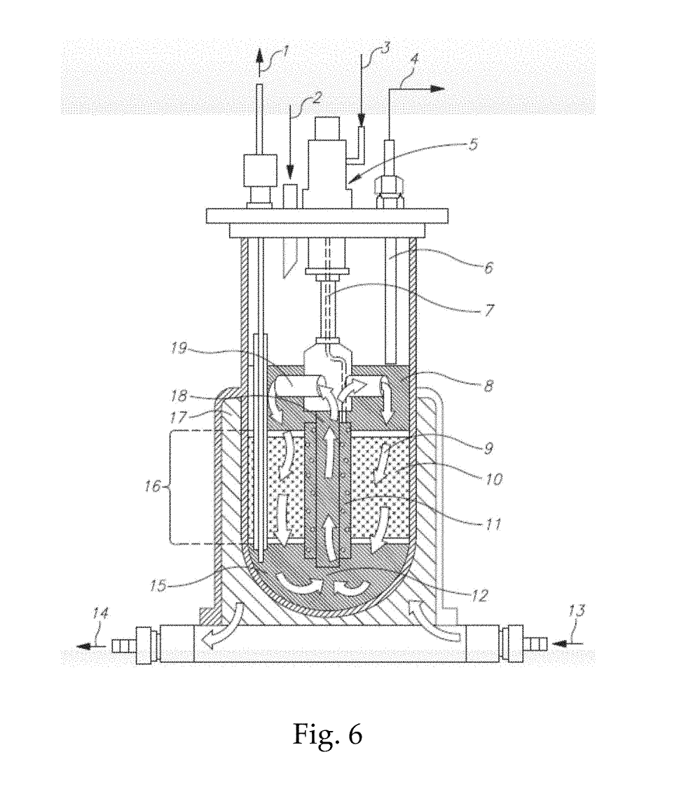

Another exemplary bioreactor, the Celligen 310 Bioreactor, is depicted in FIG. 6. A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow stirring initial rate is used to promote cell attachment, then the stirring rate is increased. Alternatively or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively or in addition, sparged gas confined to the remote chamber is absorbed by the nutrient medium, which washes over the immobilized cells. In still other embodiments, a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex, GE Healthcare), collagen-based (Cultispher, Percell), and polystyrene-based (SoloHill Engineering) microcarriers. In certain embodiments, the microcarriers are packed inside the perfused bioreactor.

In some embodiments, the carriers in the perfused bioreactor are loosely packed, for example forming a loose packed bed, which is submerged in a nutrient medium. Alternatively or in addition, the carriers are fibrous carriers that comprise an adherent material. In other embodiments, the surface of the carriers comprises an adherent material, or the surface of the carriers is adherent. In still other embodiments, the material exhibits a chemical structure such as charged surface exposed groups, which allows cell adhesion. Non-limiting examples of adherent materials which may be used in accordance with this aspect include a polyester, a polypropylene, a polyalkylene, a polyfluorochloroethylene, a polyvinyl chloride, a polystyrene, a polysulfone, a cellulose acetate, a glass fiber, a ceramic particle, a poly-L-lactic acid, and an inert metal fiber. In more particular embodiments, the material may be selected from a polyester and a polypropylene. In various embodiments, an “adherent material” refers to a material that is synthetic, or in other embodiments naturally occurring, or in other embodiments a combination thereof. In certain embodiments, the material is non-cytotoxic (or, in other embodiments, is biologically compatible). Non-limiting examples of synthetic adherent materials include polyesters, polypropylenes, polyalkylenes, polyfluorochloroethylenes, polyvinyl chlorides, polystyrenes, polysulfones, cellulose acetates, and poly-L-lactic acids, glass fibers, ceramic particles, and an inert metal fiber, or, in more specific embodiments, polyesters, polypropylenes, polyalkylenes, polyfluorochloroethylenes, polyvinyl chlorides, polystyrenes, polysulfones, cellulose acetates, and poly-L-lactic acids. Other embodiments include Matrigel™, an extra-cellular matrix component (e.g., Fibronectin, Chondronectin, Laminin), and a collagen.

In other embodiments, cells are produced using a packed-bed spinner flask. In some embodiments, the carriers are loosely packed. In more specific embodiments, the packed bed may comprise a spinner flask and a magnetic stirrer. The spinner flask may be fitted, in some embodiments, with a packed bed apparatus, which may be, in more specific embodiments, a fibrous matrix; a non-woven fibrous matrix; non-woven fibrous matrix comprising polyester; or a non-woven fibrous matrix comprising at least about 50% polyester. In more specific embodiments, the matrix may be similar to the Celligen™ Plug Flow bioreactor which is, in certain embodiments, packed with Fibra-Cel® (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the agitation speed is gradually increased, for example by starting at 40 RPM for 4 hours, then gradually increasing the speed to 120 RPM. In certain embodiments, the glucose level of the medium may be tested periodically (i.e. daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, carriers are removed from the packed bed, washed with isotonic buffer, and processed or removed from the carriers by agitation and/or enzymatic digestion.

Other embodiments of the disclosure will be apparent to those skilled in the art from consideration of the specification and practice of the present disclosure. It is intended that the specification and examples be considered as exemplary only, with a true scope and spirit of the disclosure being indicated by the following claims.

What is claimed is:

1. A method of detaching adherent cells from fibrous, three-dimensional (3D) carriers, comprising the steps of:
incubating said adherent cells with an agent that disrupts adhesion of said adherent cells to said carrier; and
subjecting said 3D carriers to a rotary motion while said 3D carriers are submerged in an aqueous solution.

2. The method of claim 1, wherein said 3D carriers are disposed within a bioreactor chamber.

3. The method of claim 1, wherein said 3D carriers and said aqueous solution are disposed within a chamber, and said rotary motion is imparted by protruding objects projecting radially from a central axis of said chamber.

4. The method of claim 3, wherein said protruding objects are rotated in a continuous rotary motion.

5. The method of claim 3, wherein said protruding objects are rotated in an oscillating rotary motion.

6. The method of claim 1, wherein said 3D carriers are packed within a basket, and said rotary motion is imparted by rotation of said basket.

7. The method of claim 6, wherein said basket is disposed within an outer container, said aqueous solution is present in said basket and said outer container, and said basket is rotated relative to said outer container.

8. The method of claim 7, wherein said basket comprises porous walls.

9. The method of claim 6, wherein said rotation is a continuous rotation.

10. The method of claim 6, wherein said rotation is an oscillating rotation.

11. The method of claim 1, wherein said agent is removed prior to said subjecting said 3D carriers to a rotary motion.

12. The method of claim 1, wherein said agent is present during said subjecting said 3D carriers to a rotary motion.

13. The method of claim 1, wherein said agent comprises a protease.

14. The method of claim 13, wherein said agent further comprises a chelator of divalent ions

15. The method of claim 1, wherein said agent comprises a chelator of divalent ions.

16. The method of claim 1, wherein said adherent cells are adherent stromal cells.

17. The method of claim 16, wherein said adherent stromal cells are placenta-derived.

18. The method of claim 17, wherein said adherent stromal cells are maternal cells.

19. The method of claim 17, wherein said adherent stromal cells are fetal cells.

20. The method of claim 16, wherein said adherent stromal cells are mesenchymal stromal cells.
